(19) United States
(12) Patent Application Publication
(10) Pub. No.: US 2017/0239657 A1
(43) Pub. Date: Aug. 24, 2017

(54) SINGLE CELL PATTERNING AND COORDINATED TRANSFER OF PATTERNED CELLS

(71) Applicant: The Methodist Hospital, Houston, TX (US)

(72) Inventors: Lidong Qin, Houston, TX (US); Kai Zhang, Houston, TX (US); Ying Li, Houston, TX (US)

(73) Assignee: The Methodist Hospital, Houston, TX (US)

(21) Appl. No.: 15/113,702

(22) PCT Filed: Jan. 23, 2015
(86) PCT No.: PCT/US15/12736

§ 371 Date: Jul. 22, 2016

Related U.S. Application Data
(60) Provisional application No. 61/930,813, filed on Jan. 23, 2014.

(51) Int. Cl. B01L 3/00; C12N 15/10

(52) U.S. Cl. CPC B01L 3/502761; C12N 15/1037; B01L 2200/0668; B01L 2300/0816; B01L 2300/0864; B01L 2400/0487; B01L 2400/086

(57) ABSTRACT

Apparatus for single cell patterning, the apparatus comprising: a structure comprising a surface channel formed therein, the surface channel being connected to an inlet and an outlet; and a cell trap disposed in the surface channel, the cell trap comprising a body defining a flow diverter for diverting flow passing by the cell trap into a wide path or a narrow path, and the body and the structure together defining a well for capturing a cell diverted by the flow diverter toward the narrow path.

### REFERENCE TO PENDING PRIOR PATENT APPLICATION

This patent application claims benefit of pending prior U.S. Provisional Patent Application Ser. No. 61/930,813, filed Jan. 23, 2014 by The Methodist Hospital and Lidong Qin et al. for SINGLE CELL PATTERNING AND BLOCK PRINTING (Attorney's Docket No. METHODIST-0810 PROV), which patent application is hereby incorporated herein by reference.

### FIELD OF THE INVENTION

This invention relates to cell isolation and manipulation in general, and more particularly to the controlled positioning of individual cells at precise locations on a substrate.

### BACKGROUND OF THE INVENTION

Current high-throughput screening of cell function and heterogeneity, and in vitro cell-cell communication studies, generally require routine generation of large-scale, single-cell arrays with high precision and efficiency, single-cell resolution, multiple cell types, and maintenance of cell viability and function. Several approaches are currently used in an effort to achieve this goal, e.g., inkjet cell printing (where individual cells are printed to a substrate in a free-flying droplet), surface engineering (where microfabrication and biochemical functionalization are combined to produce defined regions for selected cell adhesion), and physical constraints (where cell-capturing elements are used to isolate individual cells). However, for a variety of reasons, none of the approaches developed to date are completely satisfactory.

Thus, a new approach is needed for the controlled positioning of individual cells at precise locations on a substrate.

### SUMMARY OF THE INVENTION

The present invention provides a new approach for the controlled positioning of individual cells at precise locations on a substrate.

More particularly, the present invention provides a cell positioning structure having a plurality of surface channels formed therein, with the surface channels having single-cell traps disposed therein. The cell positioning structure is releasably assembled to a substrate (e.g., a commercial cell culture dish) so that the surface channels of the cell positioning structure cooperate with the substrate to provide microfluidic pathways through the assembly, with the microfluidic pathways incorporating the aforementioned single-cell traps. A slurry of cells is flowed through the microfluidic pathways of the assembly so that individual cells are captured in the single-cell traps, whereby to position the captured cells adjacent to the substrate with high positional precision. The captured cells are incubated so that the captured cells adhere to the substrate, and then the cell positioning structure is detached from the substrate, leaving individual cells disposed at precise locations on the substrate.

In one preferred form of the present invention, there is provided apparatus for single cell patterning, said apparatus comprising:

a structure comprising a surface channel formed therein, said surface channel being connected to an inlet and an outlet; and

a cell trap disposed in said surface channel, said cell trap comprising a body defining a flow diverter for diverting flow passing by said cell trap into a wide path or a narrow path, and said body and said structure together defining a well for capturing a cell diverted by said flow diverter toward said narrow path.

In another preferred form of the present invention, there is provided apparatus for single cell patterning, said apparatus comprising:

a structure comprising a surface channel formed therein, said surface channel being connected to an inlet and an outlet;

a cell trap disposed in said surface channel, said cell trap comprising a body defining a flow diverter for diverting flow passing by said cell trap into a wide path or a narrow path, and said body and said structure together defining a well for capturing a cell diverted by said flow diverter toward said narrow path; and

a substrate comprising a base, said structure being releasably mountable to said substrate so that a cell captured by said cell trap may be caused to adhere to said base.

In another preferred form of the present invention, there is provided a method for patterning individual cells on a substrate, said method comprising:

providing a structure comprising a surface channel formed therein, and comprising a cell trap disposed in said surface channel;

attaching the substrate to said structure so as to form an assembly, with said surface channel of said structure cooperating with said substrate to provide a microfluidic pathway through said assembly;

flowing a slurry of cells through said microfluidic pathway of said assembly so that an individual cell is captured in said cell trap;

causing said captured cell to adhere to said substrate; and

detaching said structure from said substrate so as to leave the captured cell disposed on said substrate.

In another preferred form of the present invention, there is provided apparatus for single cell patterning, said apparatus comprising:

a structure comprising a plurality of surface channels formed therein, said surface channels being connected to an inlet and an outlet; and

a plurality of cell traps disposed in said surface channels, each of said cell traps comprising a body defining a flow diverter for diverting flow passing by said cell trap into a wide path or a narrow path, and said body and said structure together defining a well for capturing a cell diverted by said flow diverter toward said narrow path.

In another preferred form of the present invention, there is provided apparatus for single cell patterning, said apparatus comprising:

a structure comprising a plurality of surface channels formed therein, said surface channels being connected to an inlet and an outlet;

a plurality of cell traps disposed in said surface channels, each of said cell traps comprising a body defining a flow diverter for diverting flow passing by said cell trap into a wide path or a narrow path, and said body and said structure together defining a well for capturing a cell diverted by said flow diverter toward said narrow path; and

a substrate comprising a base, said structure being releasably mountable to said substrate so that cells captured by said cell traps may be caused to adhere to said base.

In another preferred form of the present invention, there is provided a method for patterning individual cells on a substrate, said method comprising:

providing a structure comprising a plurality of surface channels formed therein, and comprising a plurality of cell traps disposed in said surface channels;

attaching the substrate to said structure so as to form an assembly, with said surface channels of said structure cooperating with said substrate to provide a microfluidic pathway through said assembly;

flowing a slurry of cells through said microfluidic pathway of said assembly so that individual cells are captured in said cell traps;

causing said captured cells to adhere to said substrate; and

detaching said structure from said substrate so as to leave the captured cells disposed on said substrate.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of a cell positioning structure formed in accordance with the present invention;

FIG. 2 is a schematic view showing surface channels and single-cell traps of the cell positioning structure shown in FIG. 1;

FIG. 3 is a schematic view showing details of a single-cell trap formed in accordance with the present invention;

FIG. 4 is a schematic view showing how a slurry of cells can be flowed through a surface channel and an individual cell captured by a single-cell trap;

FIG. 5 is a schematic view showing a selected portion of the cell positioning structure of FIG. 1 with individual cells captured in each of the single-cell traps;

FIG. 6 is a schematic view showing the cell positioning structure releasably assembled to a substrate, with the cell positioning structure and substrate being positioned against a ruler to show one possible scale, and with red dye injected into surface channels to aid in visualization;

FIG. 6A-6D are schematic views showing how the cell positioning structure may be releasably assembled to a substrate (e.g., a commercial cell culture dish), a slurry of cells flowed through the assembly so that individual cells are captured in the assembly's single-cell traps, the captured cells adhered to the substrate, and then the cell positioning structure detached from the substrate, leaving individual cells disposed at precise locations on the substrate;

FIG. 6E is a schematic view showing individual cells disposed at precise locations on the substrate;

FIGS. 7-15 are schematic views showing how the single-cell traps may be disposed in various patterns on the cell positioning structure;

FIGS. 16-18 are schematic views showing how single-cell traps may be oriented in opposite directions within the same surface channel;

FIG. 19 is a schematic view showing how single-cell traps may be oriented in opposite directions within adjacent surface channels;

FIGS. 20 and 21 are schematic views showing how a 1×N cell trap may be disposed within surface channel; and

FIGS. 22-24 are schematic views showing how N₁×N₂ cell traps may be disposed within surface channels.

### DETAILED DESCRIPTION OF THE PREFERRED EMBODIMENTS

The present invention provides a new approach for the controlled positioning of individual cells at precise locations on a substrate.

More particularly, the present invention provides a cell positioning structure having a plurality of surface channels formed therein, with the surface channels having single-cell traps disposed therein. The cell positioning structure is releasably assembled to a substrate (e.g., a commercial cell culture dish) so that the surface channels of the cell positioning structure cooperates with the substrate to provide microfluidic pathways through the assembly, with the microfluidic pathways incorporating the aforementioned single-cell traps. A slurry of cells is flowed through the microfluidic pathways of the assembly so that individual cells are captured in the single-cell traps, whereby to position the captured cells adjacent to the substrate with high positional precision. The captured cells are incubated so that the captured cells adhere to the substrate, and then the cell positioning structure is detached from the substrate, leaving individual cells disposed at precise locations on the substrate.

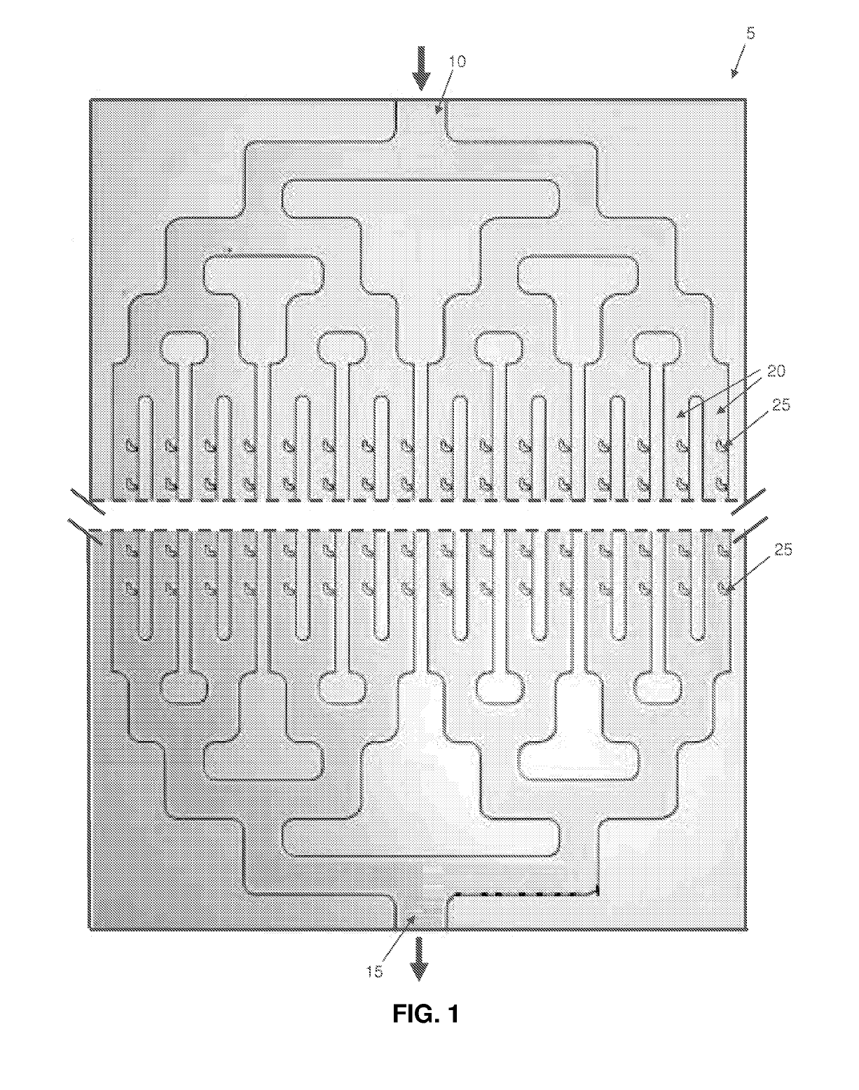

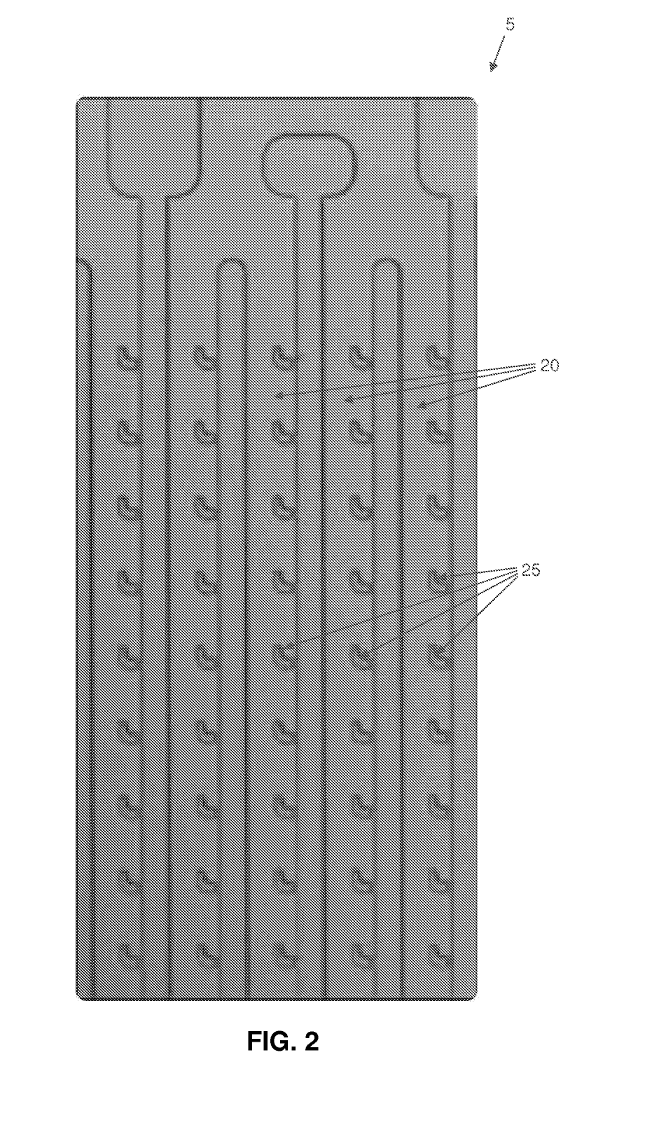

In one preferred form of the present invention, and looking now at FIGS. 1 and 2, there is provided a cell positioning structure 5 having an inlet 10, an outlet 15, and a plurality of surface channels 20 connecting inlet 10 and outlet 15. Surface channels 20 comprise a plurality of single-cell traps 25. Single cell traps 25 are disposed on cell positioning structure 5 in a predetermined, precision array, whereby to allow for the controlled positioning of individual cells in a corresponding predetermined precision array on a substrate, as will hereinafter be discussed.

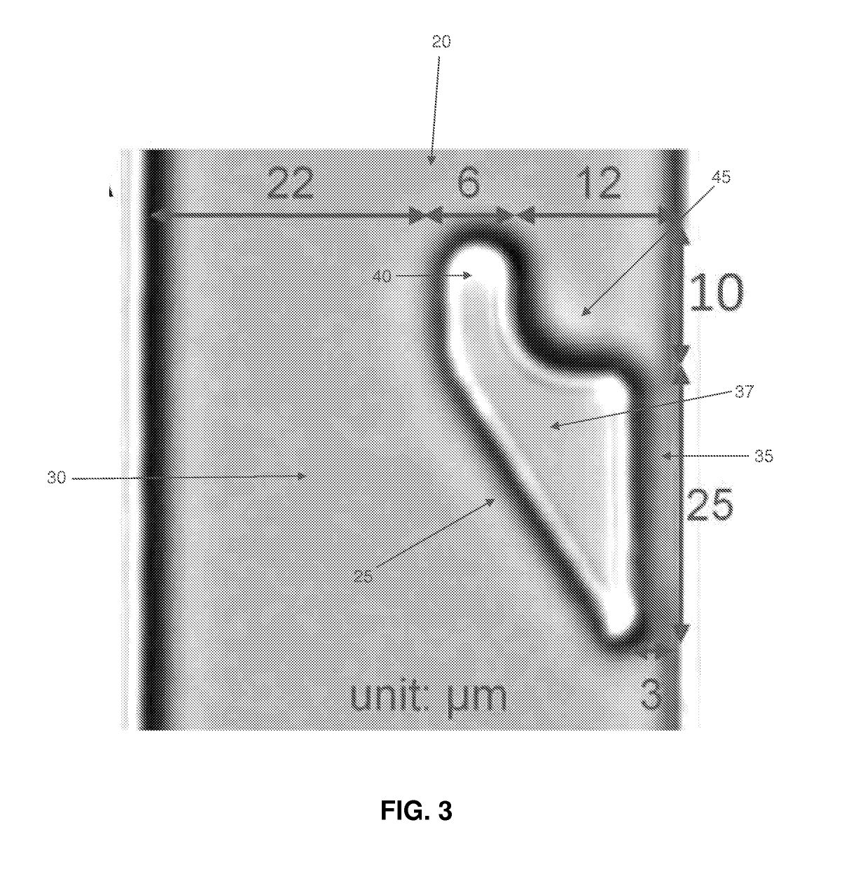

As seen in FIG. 3, each single-cell trap 25 is disposed as “an island” in surface channel 20, whereby to divide the surface channel into a wide path 30 and a narrow path 35. Wide path 30 is sized so as to be able to pass cells and fluid therethrough. Narrow path 35 is sized so as to pass only fluid therethrough. Each single-cell trap 25 comprises a body 37 comprising a flow diverter 40 and a well 45. Flow diverter 40 is spaced from the side wall of the surface channel and diverts the flow passing by single-cell trap 25 into either wide path 30 or narrow path 35. Well 45 is disposed on the downstream side of flow diverter 40 and, together with flow diverter 40 and the side wall of the surface channel, provides a seat for one individual cell.

In one preferred form of the invention, surface channel 20 may have a width of 40 μm, wide path 30 may have a width of 22 μm, narrow path 35 may have a width of 3 μm, flow diverter 40 may have a width of 6 μm and well 45 may have a width of 12 μm and a depth of 10 μm.

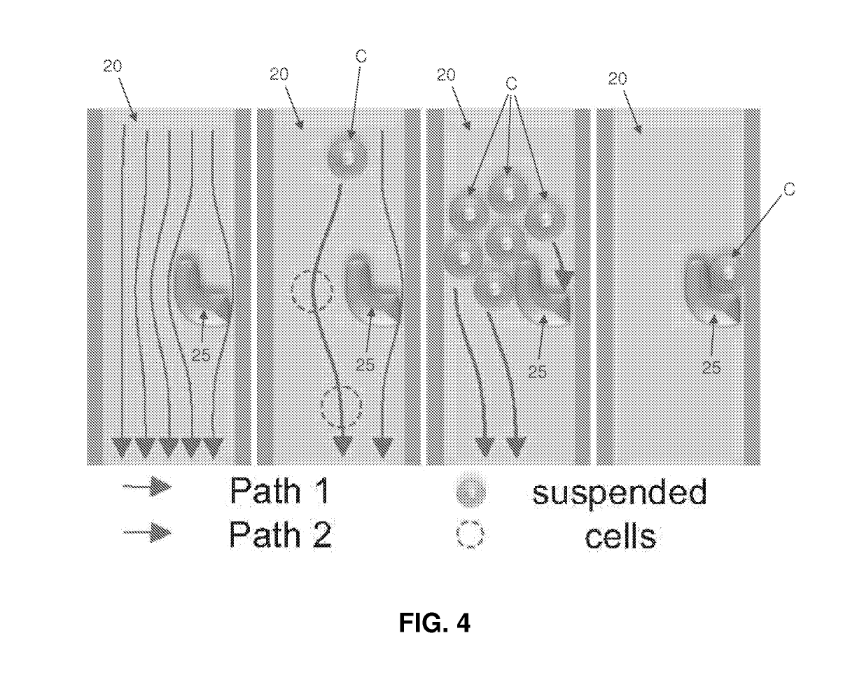

On account of the foregoing, and looking now at FIG. 4, when a slurry of cells C passes down a surface channel 20, flow diverter 40 causes some of the flow to pass down wide path 30 and some of the flow to pass down narrow path 35. Significantly, as the cells C in the slurry encounter flow diverter 40 of single-cell trap 25, most of the cells C follow wide path 30 and pass by single-cell trap 25. However, in some instances, a single cell C will be diverted into well 45 of single-cell trap 25. Note that cells C may cluster in wide path 30 adjacent to flow diverter 40 as they try to move through wide path 30, and this clustering of cells may assist in a single cell being diverted into well 45.

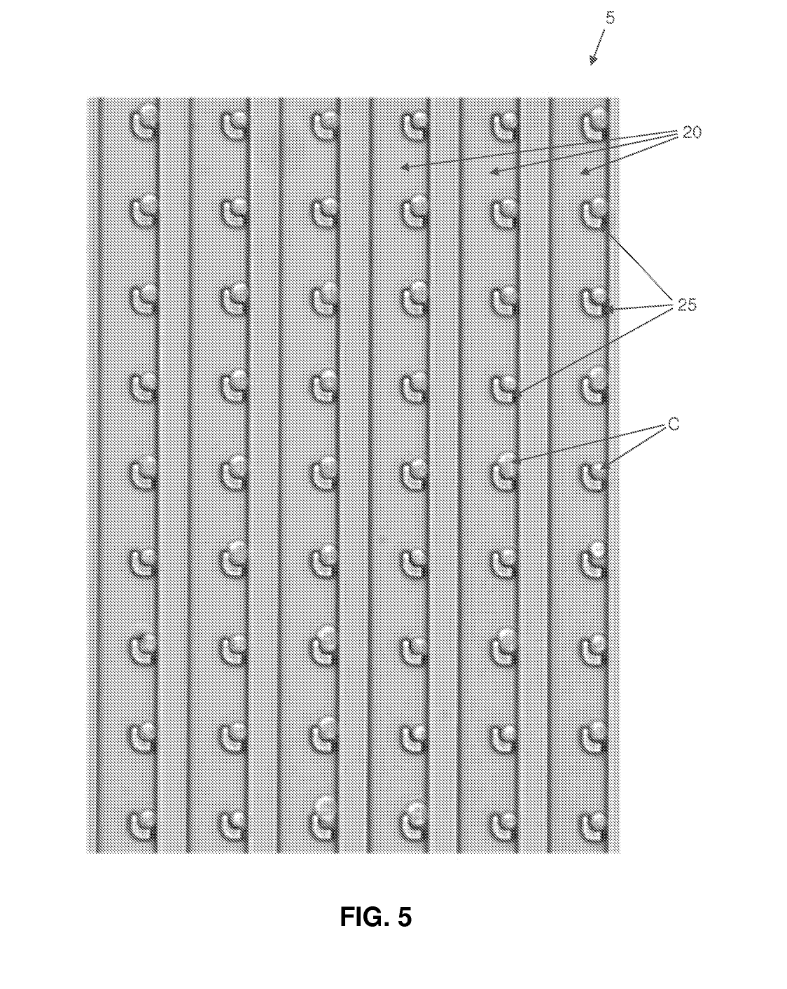

FIG. 5 shows how, after flowing a slurry of cells through a plurality of surface channels 20, individual cells C may be seated in wells 45 of single-cell traps 25. FIG. 5 also shows how, by positioning single-cell traps 25 on cell positioning structure 5 in a predetermined, precision array, the individual cells captured by single-cell traps 25 can also be positioned on cell positioning structure 5 in a predetermined, precision array.

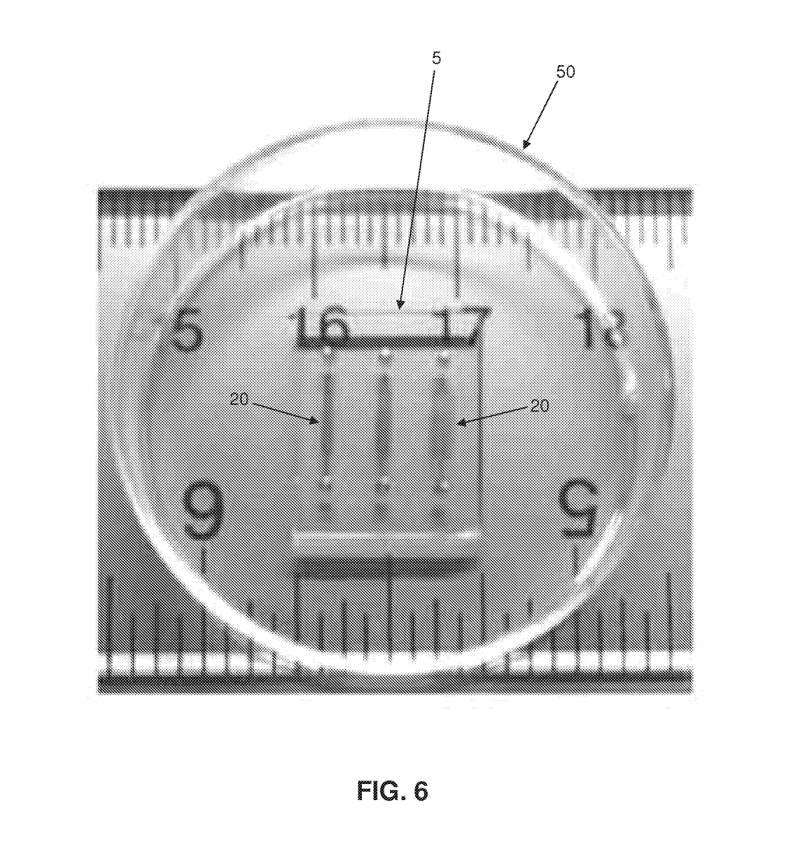

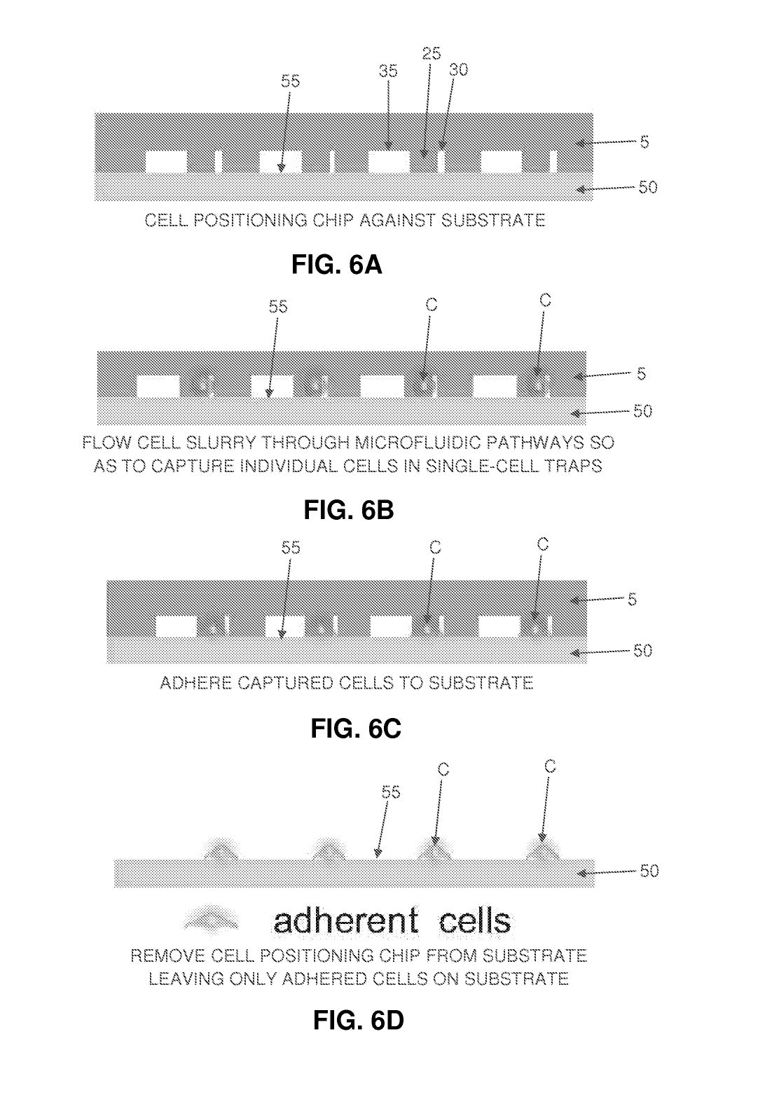

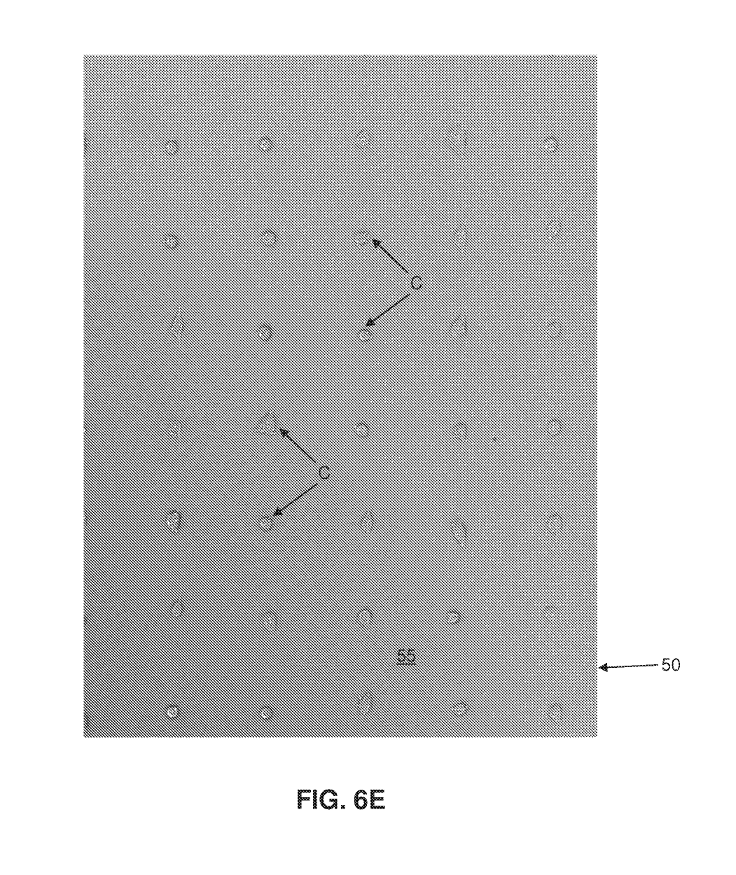

In accordance with the present invention, and looking now at FIGS. 6, 6A-6D, and 6E, cell positioning structure 5 is releasably assembled to a substrate 50 (e.g., a commercial cell culture dish) so that surface channels 20 of cell positioning structure 5 cooperate with base 55 of substrate 50 to provide microfluidic pathways through the assembly, with the microfluidic pathways incorporating the aforementioned single-cell traps 25. See FIGS. 6 and 6A. A slurry of cells is flowed through the microfluidic pathways of the assembly so that individual cells C are captured in single-cell traps 25, whereby to position the captured cells C adjacent to substrate 50 with high positional precision. See FIG. 6B. The captured cells C are then incubated so that the captured cells adhere to substrate 50. See FIG. 6C. Then cell positioning structure 5 is detached from the substrate, leaving individual cells C disposed at precise locations on the substrate. See FIGS. 6D and 6E.

If desired, captured cells C may be adhered to substrate 50 in ways other than incubation, and/or in ways in addition to incubation. By way of example but not limitation, substrate 50 may have its base 55 functionalized in ways well known in the art so that captured cells C adhere to substrate 50, e.g., by applying an adhesive substance to substrate 50.

It will be appreciated that cell positioning structure 5 and substrate 50 are configured so that, when cell positioning structure 5 is releasably assembled to substrate 50, the cells C captured in single-cell traps 25 are disposed an appropriate distance from base 55 such that the captured cells C may be adhered to, and thereafter transferred to, substrate 50.

Thus it will be seen that, by providing cell positioning structure 5 with a plurality of surface channels 20 having a plurality of single-cell traps 25 disposed therein, with single-cell traps 25 being disposed on cell positioning structure 5 in a predetermined, precision array, individual cells may be precisely positioned on substrate 50 in a corresponding predetermined precision array.

In one preferred form of the invention, cell positioning structure 5 may be fabricated in the following manner. The design is drawn with CAD software and printed out as glass photomasks (Photo Sciences Inc.). Polydimethylsiloxane (PDMS) molds are then fabricated by standard photolithography and elastomer molding. In one preferred form of the invention, SPR 220-7 positive photoresist (MicroChem Corp.) is used to fabricate 12 μm thick channels and SU-8 3025 negative photoresist (MicroChem Corp.) is used to fabricate 17-μm thick channels. SPR 220-7 photoresist is spin-coated onto a 4 inch silicon wafer (Silicon Quest International Inc.) at 1,500 rpm for 40 seconds to form a layer approximately 12 μm thick. After baking at 75° C. for 3 minutes, and then at 115° C. for 5 minutes, the wafer is cooled, exposed to UV light for 7 seconds, and developed. SU-8 3025 photoresist is spin-coated onto a 4 inch silicon wafer at 4,000 rpm for 60 seconds to form a layer approximately 17 μm inch thickness. After soft baking at 65° C. for 2 minutes and then at 95° C. for 10 minutes, the wafer is cooled and exposed to UV light for 6 seconds. It is then heated for post-exposure baking at 65° C. for 1 minute and then at 95° C. for 3 minutes. After cooling down, the wafer is developed and heated for hard baking at 135° C. for 20 minutes. Finally PDMS (10A:1B, Dow Corning Corp.) is poured onto the photoresist mold and heated at 75° C. for 30 minutes. After curing, the PDMS is peeled off, cut to the appropriate size, and then punched to form cell positioning structure 5.

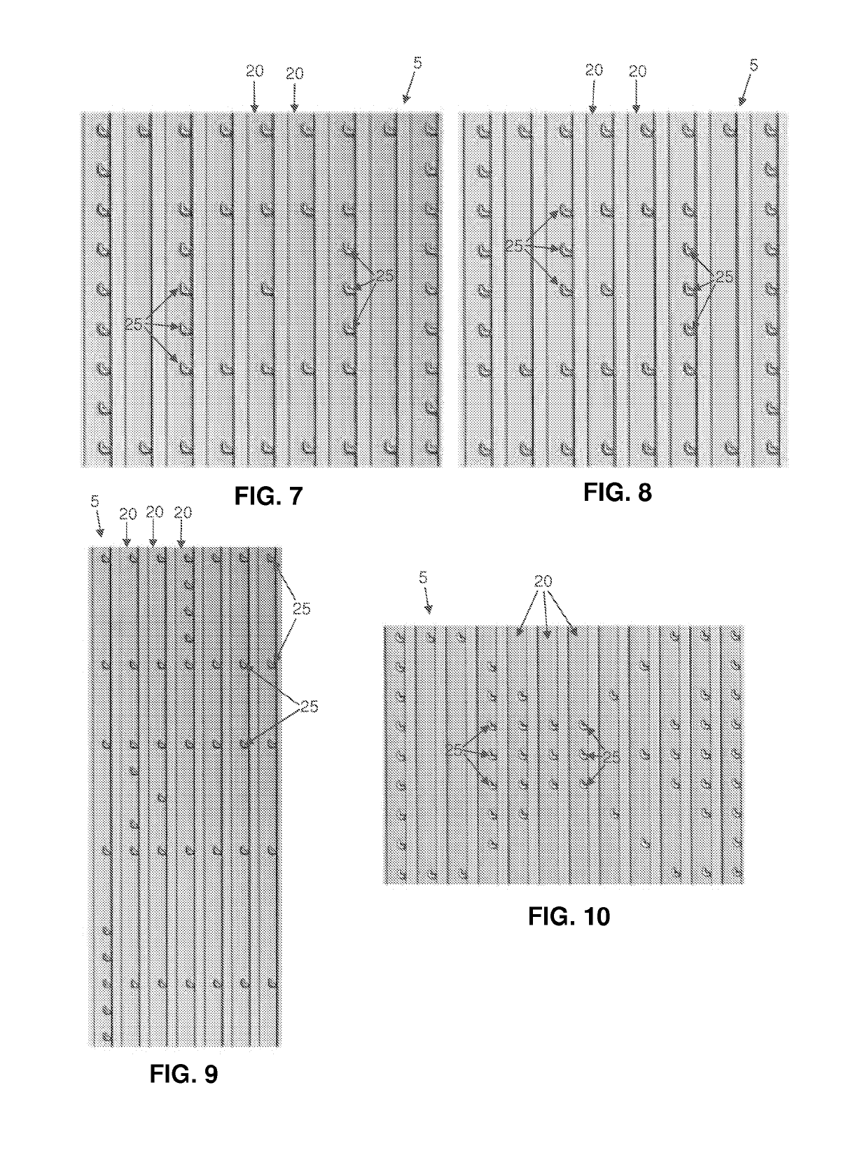

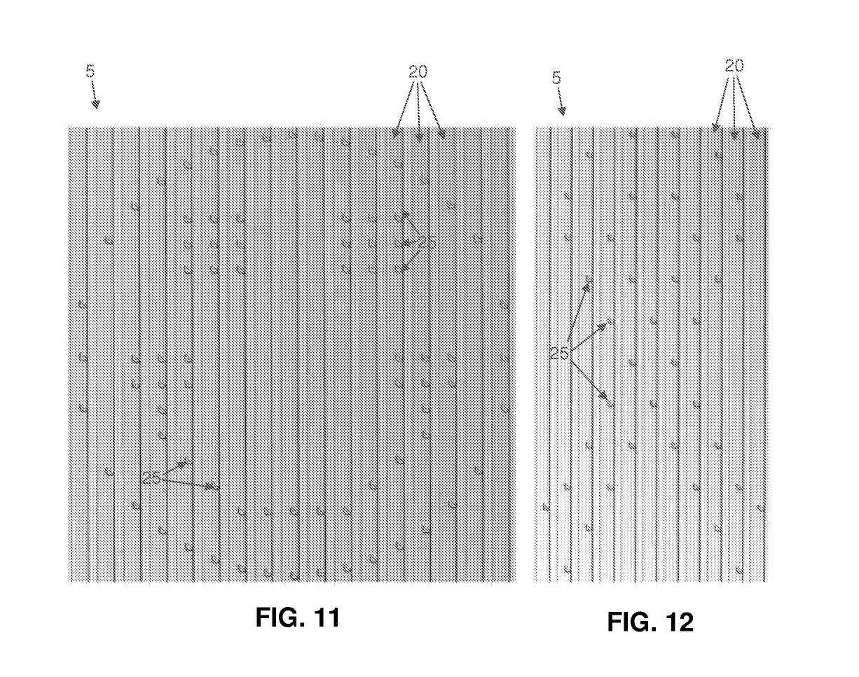

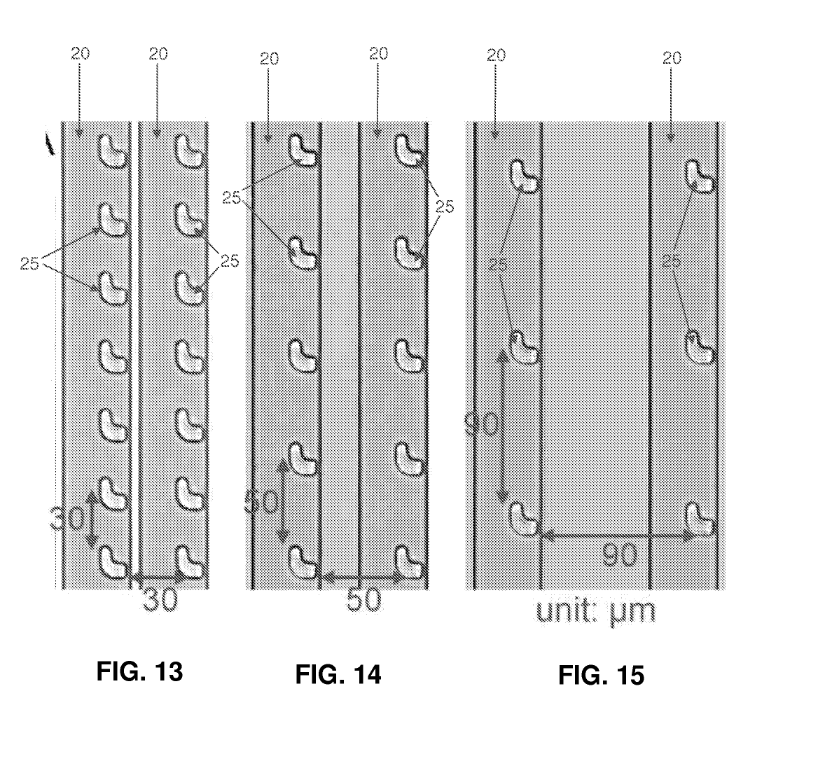

It will be appreciated that, by varying the disposition of surface channels 20 and single-cell traps 25 on cell positioning structure 5, the disposition of individual cells on substrate 50 may be similarly varied. See, for example, FIGS. 7-15, which show various dispositions of surface channels 20 and single-cell traps 25 on cell positioning structure 5.

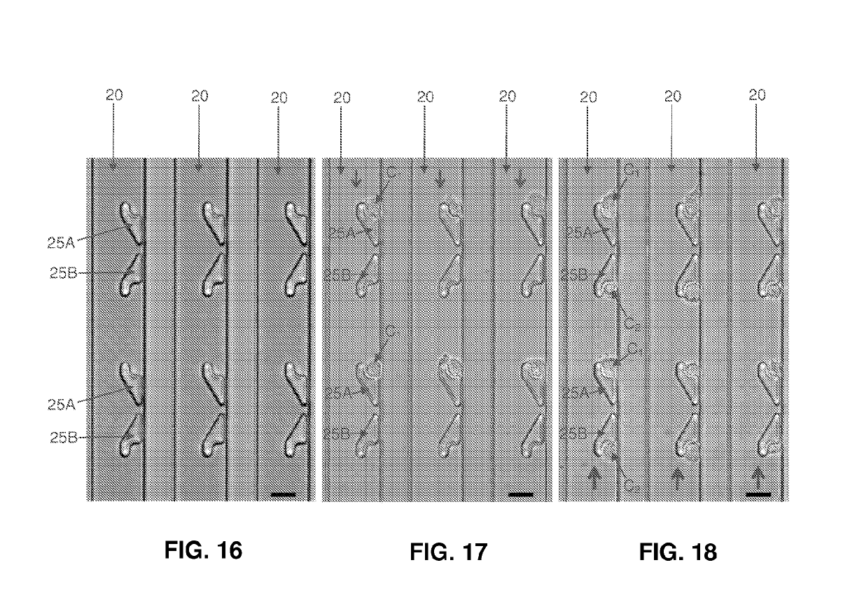

FIGS. 16-18 are schematic views showing how single-cell traps 25 may also be oriented in opposite directions within the same surface channel 20. More particularly, with the embodiments shown in FIGS. 1-15, the single-cell traps 25 disposed in a particular surface channel 20 are shown as all being oriented in the same direction (i.e., with their flow diverter 40 oriented towards inlet 10). However, if desired, and looking now at FIGS. 16-18, single-cell traps 25 may be oriented in opposite directions within the same surface channel 20. As a result of this construction, by passing a first cell slurry containing a first cell type C₁ through a surface channel 20 in a first direction, individual C₁ cells may be captured in single-cell traps 25A (see FIG. 17); and by passing a second cell slurry containing a second cell type C₂ through the same surface channel 20 in the opposite direction, individual C₂ cells may be captured in single-cell traps 25B. In this way, after the C₁ and C₂ cells are adhered to substrate 50 (e.g., by incubation), the C₁ and C₂ cells will be disposed on substrate 50 with a predetermined spatial relationship to one another (i.e., the spatial relationship which is defined by the spatial relationship of the single-cell traps 25A to the single-cell traps 25B). Such an arrangement can be useful when two different cell types must be placed in controlled disposition relative to one another, e.g., such as when studying cell-cell communications.

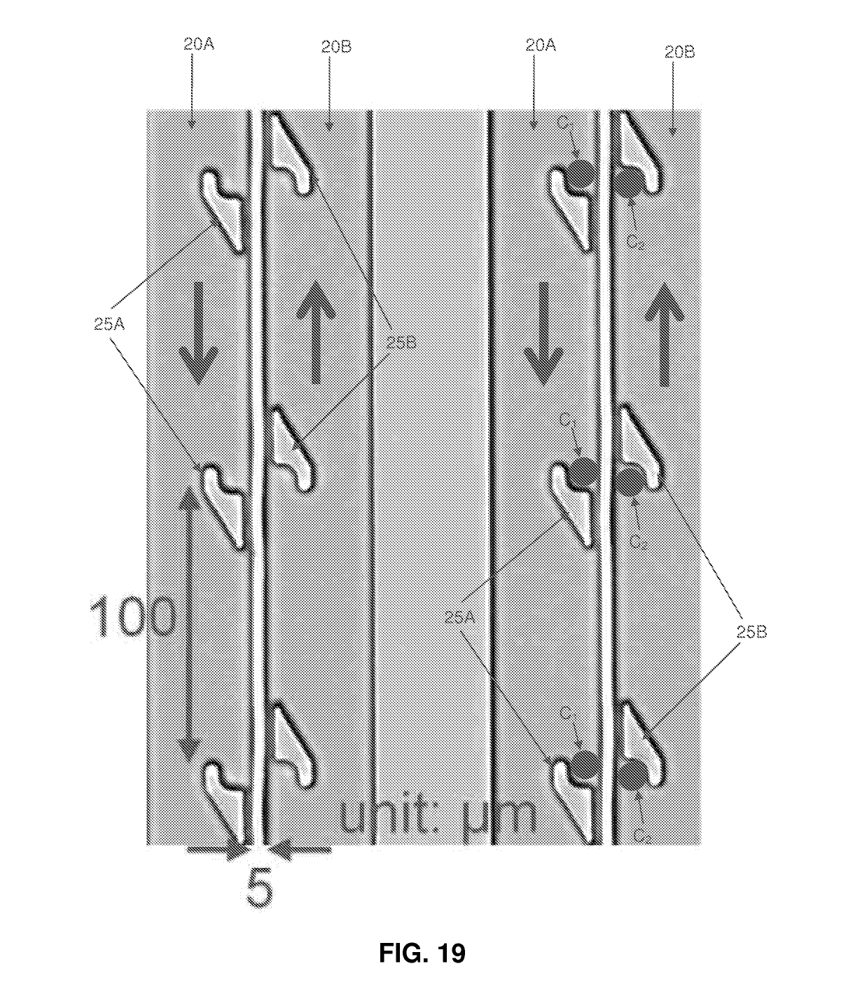

FIG. 19 is a schematic view showing how single-cell traps 25 may also be oriented in opposite directions within adjacent surface channels 20. More particularly, in the embodiments shown in FIGS. 1-15, the single-cell traps 25 disposed in adjacent surface channels 20 are shown as all being oriented in the same direction (i.e., with their flow diverter 40 oriented towards inlet 10). However, if desired, and looking now at FIG. 19, single-cell traps 25 may be disposed in opposite directions within adjacent surface channels 20. As a result of this construction, by passing a first cell slurry containing a first cell type C₁ through a surface channel 20A in a first direction, individual C₁ cells may be captured in single-cell traps 25A; and by passing a second cell slurry containing a second cell type C₂ through an adjacent surface channel 20B in the opposite direction, individual C₂ cells may be captured in single-cell traps 25B. In this way, after the C₁ and C₂ cells are adhered to substrate 50 (e.g., by incubation), the C₁ and C₂ cells will be disposed on substrate 50 with a predetermined spatial relationship to one another (i.e., the spatial relationship which is defined by the spatial relationship of the single-cell traps 25A to the single-cell traps 25B). Such an arrangement can be useful when two different cell types must be placed in controlled disposition relative to one another, e.g., such as when studying cell-cell communications.

It is also possible to practice the present invention with cell traps capable of capturing more than one cell. More particularly, in the embodiments shown in FIGS. 1-19, the cell traps 25 disposed in surface channels 20 of cell positioning structure 5 are all “single-cell traps”, in the sense that each cell trap is specifically configured to trap a single cell. However, if desired, it is possible to configure the cell traps to capture more than one cell.

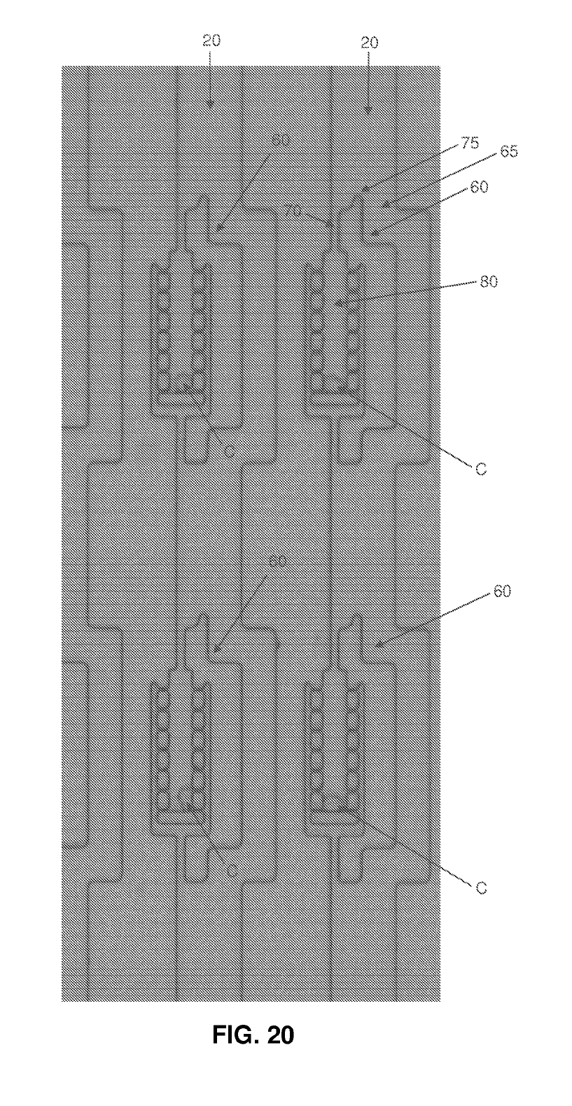

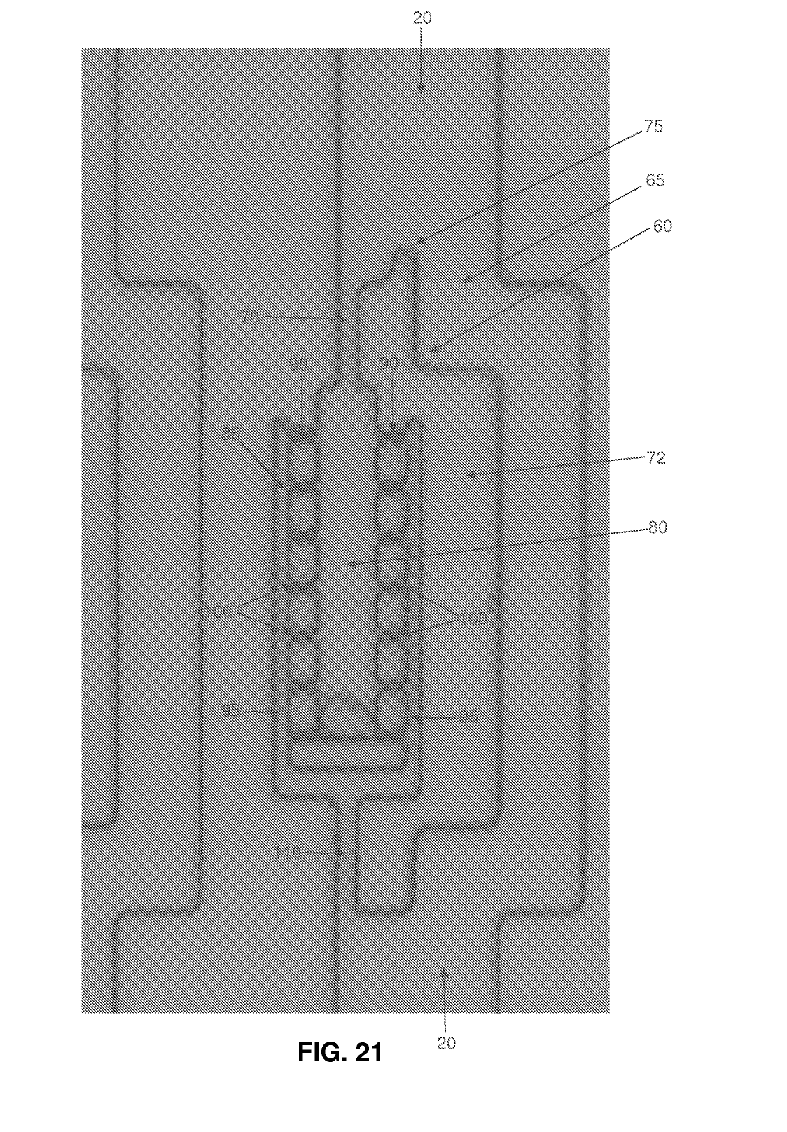

By way of example but not limitation, and looking now at FIGS. 20 and 21, there is shown a 1×N cell trap 60. For the purposes of the present invention, a “1×N” cell trap is intended to mean a cell trap configured to be “1” cell wide and “N” cells long. Each 1×N cell trap 60 is disposed in a surface channel 20 formed in cell positioning structure 5. In one preferred form of the invention, each 1×N cell trap 60 is disposed as “an island” in surface channel 20, whereby to divide the surface channel into a wide path 65 and a narrow path 70. Wide path 65 is sized so as to be able to pass cells and fluid therethrough. Narrow path 70 is sized so as to pass cells and fluid therethrough, but narrow path 70 is sized so that only one cell at a time can pass therethrough. Each 1×N cell trap 60 comprises a body 72 defining a flow diverter 75 and a 1×N well 80. Flow diverter 75 is spaced from the side wall of the surface channel and diverts the flow passing by 1×N cell trap 60 into either wide path 65 or narrow path 70. Well 80 is disposed on the downstream side of flow diverter 75 and, together with flow diverter 75 and the side wall of the surface channel, provides a seat to accommodate a plurality of cells when those cells are stacked in a 1×N array.

In order to permit continuous fluid flow into well 80 (i.e., via narrow path 70), well 80 preferably comprises a filter 85. Filter 85 generally comprises walls 90 spaced from body 72 and the side wall of the surface channel so as to provide gaps 95 therebetween. A plurality of openings 100 are formed in walls 90 so as to provide fluid communication between well 80 and gaps 95. Gaps 95 communicate with an outlet 110 which is in fluid communication with surface channel 20. By virtue of this construction, fluid can flow through narrow path 70, into well 80, through openings 100, into gaps 95, through outlet 110 and back into surface channel 20. However, if a cell is diverted into narrow path 70 and into well 80, the cell will be too large to pass through openings 100 and into gaps 95, and thus will be retained in well 80.

On account of the foregoing, when a slurry of cells passes down a surface channel 20, flow diverter 75 causes some of the flow to pass down wide path 65 and some of the flow to pass down narrow path 70. Significantly, as the cells in the slurry encounter flow diverter 75 of 1×N cell trap 60, most of the cells follow wide path 65 and pass by 1×N cell trap 60. However, in some instances, a single cell C will be diverted into well 80 of 1×N cell trap 60, whereby to form a 1×N array of cells. Again, note that cells C may cluster in wide path 65 adjacent to flow diverter 75 as they try to move through wide path 65, and this clustering of cells may assist in one cell at a time being diverted into well 80.

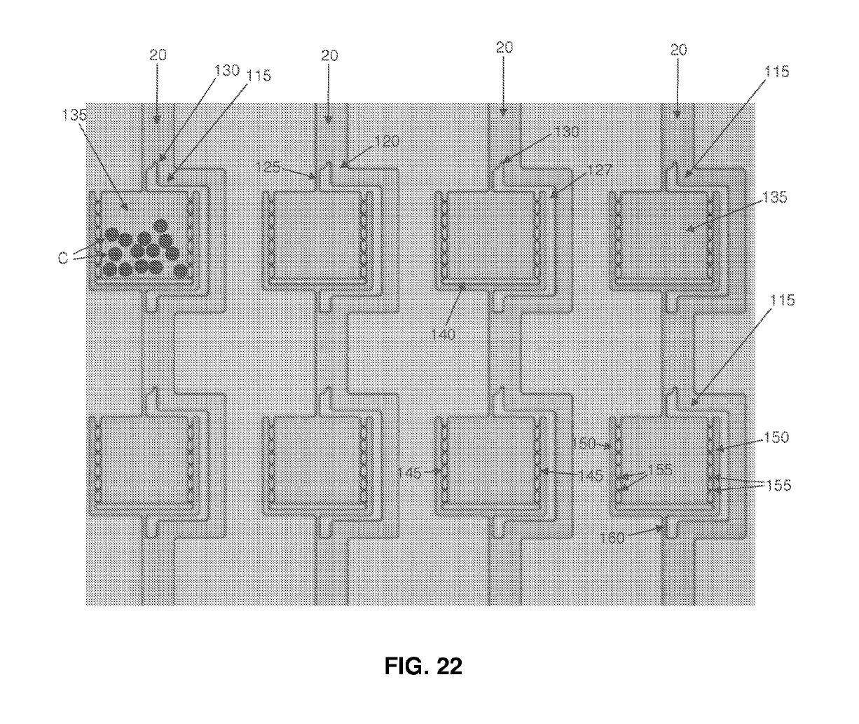

By way of further example but not limitation, and looking now at FIG. 22, there is shown an N₁×N₂ cell trap 115. For the purposes of the present invention, an “N₁×N₂” cell trap is intended to mean a cell trap configured to be “N₁” cells wide and “N₂” cells long, where the cell trap has a rectangular body—and a maximum of “N₁” cells wide and “N₂” cells long where the cell trap has a non-rectangular body such as a circle or triangle. Each N₁×N₂ cell trap 115 is disposed as “an island” in surface channel 20, whereby to divide the surface channel into a wide path 120 and a narrow path 125. Wide path 120 is sized so as to be able to pass cells and fluid therethrough. Narrow path 125 is sized so as to pass cells and fluid therethrough, but narrow path 125 is sized so that only one cell at a time can pass therethrough. Each N₁×N₂ cell trap 115 comprises a body 127 defining a flow diverter 130 and a N₁×N₂ well 135. Flow diverter 130 is spaced from the side wall of the surface channel and diverts the flow passing by N₁×N₂ cell trap 115 into either wide path 120 or narrow path 125. N₁×N₂ well 135 is sized to accommodate a plurality of cells when those cells are stacked in a N₁×N₂ array.

In order to permit continuous fluid flow into well 135 (i.e., via narrow path 125), well 135 preferably comprises a filter 140. Filter 140 generally comprises walls 145 spaced from body 127 and the side wall of the surface channel so as to provide gaps 150 therebetween. A plurality of openings 155 are formed in walls 145 so as to provide fluid communication between well 135 and gaps 150. Gaps 150 communicate with an outlet 160 which is in fluid communication with surface channel 20. By virtue of this construction, fluid can flow through narrow path 125, into well 135, through openings 155, into gaps 150, through outlet 160 and back into surface channel 20. However, if a cell is diverted into narrow path 125 and into well 135, the cell will be too large to pass through openings 155 and into gaps 150 and thus will be retained in well 135.

On account of the foregoing, when a slurry of cells passes down a surface channel 20, flow diverter 130 causes some of the flow to pass down wide path 120 and some of the flow to pass down narrow path 125. Significantly, as cells in the slurry encounter flow diverter 130 of N₁×N₂ cell trap 115, most of the cells follow wide path 120 and pass by N₁×N₂ cell trap 115. However, in some instances, a single cell will be diverted into well 135 of N₁×N₂ cell trap 115, whereby to form an N₁×N₂ array of cells. Again, note that cells may cluster in wide path 120 adjacent to diverter 135 as they try to move through wide path 120, and this clustering of cells may assist in a single cell at a time being diverted into well 135.

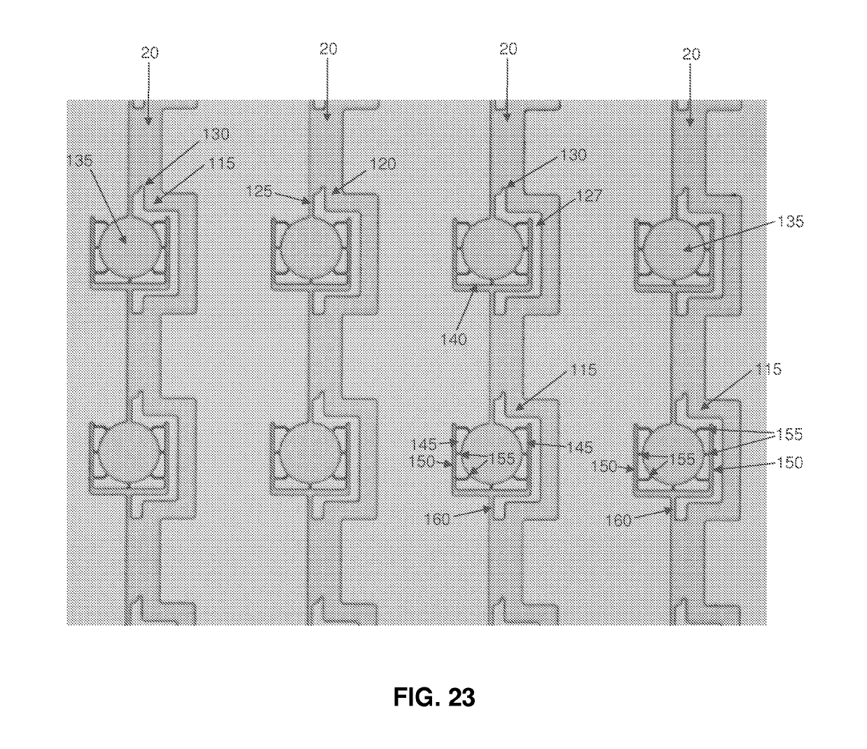

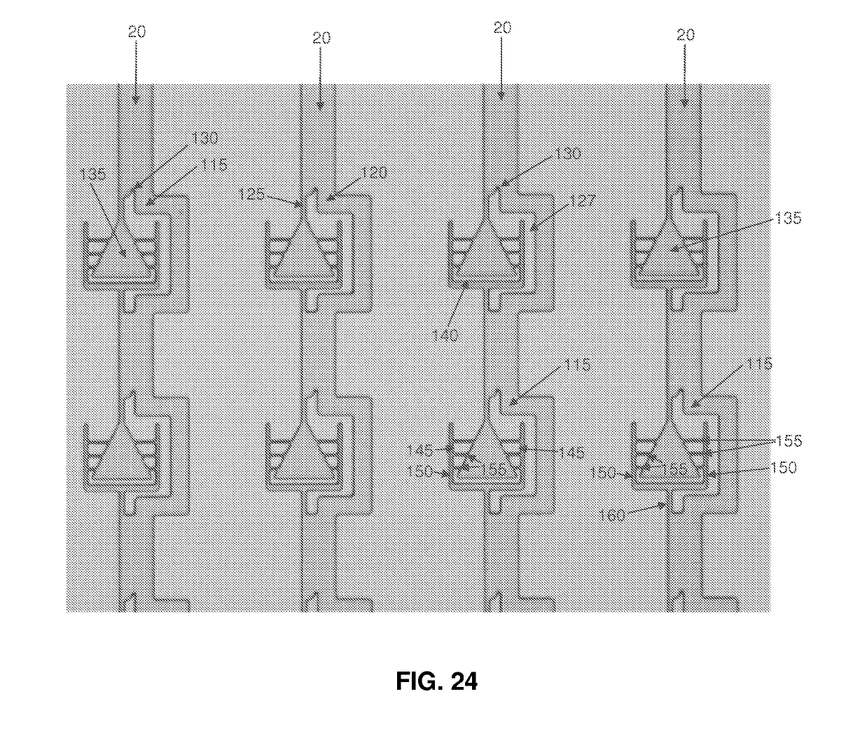

As seen in FIGS. 23 and 24, the geometry of N₁×N₂ cell trap 115 may be varied so as to influence the geometry of the cell mass captured in N₁×N₂ cell trap 115 and thereafter adhered to substrate 50.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for single cell patterning, said apparatus comprising:
a structure comprising a surface channel formed therein, said surface channel being connected to an inlet and an outlet; and
a cell trap disposed in said surface channel, said cell trap comprising a body defining a flow diverter for diverting flow passing by said cell trap into a wide path or a narrow path, and said body and said structure together defining a well for capturing a cell diverted by said flow diverter toward said narrow path.

2. Apparatus according to claim 1 wherein said surface channel comprises a plurality of cell traps.

3. Apparatus according to claim 1 wherein said structure comprises a plurality of surface channels.

4. Apparatus according to claim 3 wherein each surface channel comprises a plurality of cell traps.

5. Apparatus according to claim 1 wherein said cell trap is configured to capture a single cell.

6. Apparatus according to claim 5 wherein said body of said cell trap is separated from said structure by a distance small enough to prevent a cell from passing therethrough.

7. Apparatus according to claim 5 wherein said cell trap is configured to receive a single cell.

8. Apparatus according to claim 1 wherein said cell trap is configured to capture a plurality of cells.

9. Apparatus according to claim 8 wherein said cell trap is configured to capture a plurality of cells one at a time.

10. Apparatus according to claim 9 wherein said cell trap is configured to hold the captured cells in a 1×N array.

11. Apparatus according to claim 9 wherein said cell trap is configured to hold the captured cells in an N₁×N₂ array.

12. Apparatus according to claim 1 wherein said surface channel comprises a plurality of cell traps, and further wherein at least some of said cell traps have their diverters directed in a common direction.

13. Apparatus according to claim 1 wherein said structure comprises a plurality of surface channels, and further wherein at least some of said cell traps in adjacent surface channels have their diverters directed in opposite directions.

14. Apparatus according to claim 1 further comprises a substrate for receiving a cell captured by said cell trap.

15. Apparatus according to claim 14 wherein said substrate is releasably mounted to said structure.

16. Apparatus according to claim 14 wherein said substrate comprises a cell culture dish.

17. Apparatus according to claim 14 wherein said substrate receives a cell captured by said cell trap after incubation.

18. Apparatus according to claim 14 wherein said structure comprises a plurality of surface channels each comprising a plurality of cell traps, wherein said cell traps are arranged on said structure in a predetermined, precision array, and further wherein said substrate receives the cells captured by said cell traps as a function of said predetermined, precision array.

19. Apparatus for single cell patterning, said apparatus comprising:
a structure comprising a surface channel formed therein, said surface channel being connected to an inlet and an outlet;
a cell trap disposed in said surface channel, said cell trap comprising a body defining a flow diverter for diverting flow passing by said cell trap into a wide path or a narrow path, and said body and said structure together defining a well for capturing a cell diverted by said flow diverter toward said narrow path; and
a substrate comprising a base, said structure being releasably mountable to said substrate so that a cell captured by said cell trap may be caused to adhere to said base.

20. Apparatus according to claim 19 wherein a cell captured by said cell trap may be caused to adhere to said base by incubating said cell.

21. A method for patterning individual cells on a substrate, said method comprising:
providing a structure comprising a surface channel formed therein, and comprising a cell trap disposed in said surface channel;
attaching the substrate to said structure so as to form an assembly, with said surface channel of said structure cooperating with said substrate to provide a microfluidic pathway through said assembly;
flowing a slurry of cells through said microfluidic pathway of said assembly so that an individual cell is captured in said cell trap;
causing said captured cell to adhere to said substrate; and
detaching said structure from said substrate so as to leave the captured cell disposed on said substrate.

22. A method according to claim 21 wherein said cell trap comprises a body defining a flow diverter for diverting flow passing by said cell trap into a wide path or a narrow path, and said body and said structure together defining a well for capturing a cell diverted by said flow diverter toward said narrow path.

23. A method according to claim 22 wherein said surface channel comprises a plurality of cell traps.

24. A method according to claim 22 wherein said structure comprises a plurality of surface channels.

25. A method according to claim 24 wherein each surface channel comprises a plurality of cell traps.

26. A method according to claim 22 wherein said cell trap is configured to capture a single cell.

27. A method according to claim 26 wherein said body of said cell trap is separated from said structure by a distance small enough to prevent a cell from passing therethrough.

28. A method according to claim 26 wherein said cell trap is configured to receive a single cell.

29. A method according to claim 22 wherein said cell trap is configured to capture a plurality of cells.

30. A method according to claim 29 wherein said cell trap is configured to capture a plurality of cells one at a time.

31. A method according to claim 30 wherein said cell trap is configured to hold the captured cells in a 1×N array.

32. A method according to claim 30 wherein said cell trap is configured to hold the captured cells in an N₁×N₂ array.

33. A method according to claim 22 wherein said surface channel comprises a plurality of cell traps, and further wherein at least some of said cell traps have their diverters directed in a common direction.

34. A method according to claim 22 wherein said structure comprises a plurality of surface channels, and further wherein at least some of said cell traps in adjacent surface channels have their diverters directed in opposite directions.

35. A method according to claim 21 wherein said substrate comprises a cell culture dish.

36. A method according to claim 21 wherein said captured cell is caused to adhere to said substrate by incubation.

37. Apparatus according to claim 21 wherein said structure comprises a plurality of surface channels each comprising a plurality of cell traps, wherein said cell traps are arranged on said structure in a predetermined, precision array, and further wherein said substrate receives the cells captured by said cell traps as a function of said predetermined, precision array.

38. Apparatus for single cell patterning, said apparatus comprising:
a structure comprising a plurality of surface channels formed therein, said surface channels being connected to an inlet and an outlet; and
a plurality of cell traps disposed in said surface channels, each of said cell traps comprising a body defining a flow diverter for diverting flow passing by said cell trap into a wide path or a narrow path, and said body and said structure together defining a well for capturing a cell diverted by said flow diverter toward said narrow path.

39. Apparatus for single cell patterning, said apparatus comprising:
a structure comprising a plurality of surface channels formed therein, said surface channels being connected to an inlet and an outlet;
a plurality of cell traps disposed in said surface channels, each of said cell traps comprising a body defining a flow diverter for diverting flow passing by said cell trap into a wide path or a narrow path, and said body and said structure together defining a well for capturing a cell diverted by said flow diverter toward said narrow path; and
a substrate comprising a base, said structure being releasably mountable to said substrate so that cells captured by said cell traps may be caused to adhere to said base.

40. A method for patterning individual cells on a substrate, said method comprising:
providing a structure comprising a plurality of surface channels formed therein, and comprising a plurality of cell traps disposed in said surface channels;
attaching the substrate to said structure so as to form an assembly, with said surface channels of said structure cooperating with said substrate to provide a microfluidic pathway through said assembly;
flowing a slurry of cells through said microfluidic pathway of said assembly so that individual cells are captured in said cell traps;
causing said captured cells to adhere to said substrate; and
detaching said structure from said substrate so as to leave the captured cells disposed on said substrate.
